(19) United States
(12) Patent Application Publication
(10) Pub. No.: US 2010/0285156 A1
(43) Pub. Date: Nov. 11, 2010

(54) COMPOSITION FOR PREVENTING AND TREATING METABOLIC DISEASES COMPRISING THE EXTRACT OF LYSIMACHIAE FOENUM-GRAECI HERBA

(75) Inventors: Jong-Bae Seo, Seoul (KR); Jae-Young Park, Seoul (KR); Sun-Mi Choi, Gyeonggi-Do (KR); Sang-Wook Park, Seoul (KR); Sung-Sik Choe, Seoul (KR); Eun-Wook Choi, Seoul (KR); Dong-Seung Seen, Gyeonggi-Do (KR); Tae-Gyu Lee, Seoul (KR)

Correspondence Address: Edwards Angell Palmer & Dodge LLP, P.O. Box 55874, Boston, MA, 02205, US

(73) Assignee: BRNSCIENCE CO., LTD, Seoul (KR)

(21) Appl. No.: 12/811,056

(22) PCT Filed: Dec. 28, 2008
(86) PCT No.: PCT/KR2008/007706

§ 371 Date: Jun. 28, 2010

(30) Foreign Application Priority Data
Dec. 28, 2007 (KR) 10-2007-0140988

(51) Int. Cl. A61K 36/185; A61P 3/10; A61P 3/06; A61P 1/16; A61P 9/10

(52) U.S. Cl. 424/725

(57) ABSTRACT

Disclosed are raw material, functional foods, and herb medicines for preventing and treating metabolic diseases, which comprise a Lysimachiae Foenum-Graeci Herba extract as an effective ingredient. The Lysimachiae Foenum-Graeci Herba extract have an activity of reducing blood glucose levels, triglycerides and cholesterol levels, AST and ALT levels, and fat in the liver, and thus can be effectively used as a hepatic protector and a remedy for preventing and treating various metabolic diseases including diabetes, high blood pressure, fatty liver, cardiovascular diseases, and hyperlipidemia.

### TECHNICAL FIELD

The present invention relates to a composition for preventing and treating metabolic diseases, which comprises a Lysimachiae Foenum-Graeci Herba extract as an effective ingredient, and more particularly to raw materials, functional foods, and herb medicines for use as a hepatic protector and a remedy for preventing and treating a variety of metabolic diseases, including diabetes, high blood pressure, fatty liver, cardiovascular diseases, and hyperlipidemia, by reducing blood glucose levels, triglycerides and cholesterol levels, AST and ALT levels, and liver fat.

### BACKGROUND ART

In recent years, as the standard of living has increased according to economic growth, the health environment has been improved, and eating habits have changed in the direction of frequent junk food intake and a meat-centered diet, which results in an excessive accumulation of caloric energy in the body. However, since a reduction in caloric energy consumption, caused by lack of exercise, is added to such a change in the contemporary eating habits, there is a tendency that obese population rapidly increases. In this way, caloric energy excessively accumulated in the body emerges in the form of various diseases, as well as obesity, and examples of such diseases include metabolic diseases and metabolic syndrome, such as diabetes, hyperlipidemia, and fatty liver.

Diabetes is a kind of systemic metabolic disease resulting from genetic and environmental factors, and refers to a state in which an abnormally high glucose concentration in blood is caused by absolute and relative insulin deficiency in the body. The complications of diabetes include hypoglycemia, ketoacidosis, hyperosmolar coma, macrovascular complications, diabetic retinopathy, diabetic neuropathy, diabetic nephropathy.

Hyperlipidemia is a general term for hypercholesterolemia and hypertriglycerides, and refers to a state in which cholesterol (240 mg/dl or more) and triglycerides (200 mg/dl or more) increase above normal ranges due to disorder of lipoprotein and lipid metabolism. Hyperlipidemia is largely classified into two types, that is, primary hyperlipidemia resulting from genetic abnormalities and secondary hyperlipidemia resulting from other diseases, such as diabetes, or drugs. Hyperlipidemia does not develop any particular symptom, but has a problem in that increases in cholesterol and triglycerides in blood may cause arteriosclerosis, high blood pressure, cardiovascular diseases, and so forth.

Fatty liver refers to a state in which fat is accumulated in hepatocytes. Since accumulated fat itself has no toxicity to hepatocytes, there is no symptom and liver functions are normal or slightly deteriorate in most cases where fatty liver is not serious. However, if fatty liver becomes serious to enlarge chunks of fat in hepatocytes, the functions of the hepatocytes including nuclei deteriorate. That is, fat accumulated in hepatocytes oppresses microvessels and lymph glands between the hepatocytes to impede blood and lymph circulations in the liver. As a result of this, proper oxygen and nutrition cannot be supplied to the hepatocytes, and thus liver functions deteriorate.

Cardiovascular diseases refer to cardiac and vascular abnormalities, and include heart diseases, diseases of major blood vessels, such as aortae, and peripheral vascular diseases of sub-organs/sub-tissues. More specially, cardiovascular diseases cover heart and vascular diseases; the former of which include cardiac failure, hypertensive heart diseases, arrhythmia, valvular diseases, congenital heart diseases, and cardiomyopathy, and the latter of which include cerebrovascular accident and peripheral vascular diseases.

These diseases are caused by increased arteriosclerotic factors due to a rise in cholesterol, vascular endothelial cell damage due to LDL oxidation, blood flow disturbance due to blood clotting, and so forth, and may be worsened by hyperlipidemia, arteriosclerosis, etc. In other words, lipid metabolism and lipid concentration in blood largely affect the onset and progress of the cardiovascular diseases. More specially, cardiovascular diseases may be improved by preventing LDL oxidation to lower LDL cholesterol levels and increasing HDL cholesterol levels to reduce arteriosclerotic factors.

That is, the onset of various cardiovascular diseases resulting from hyperlipidemia and blood dysfunction may be essentially prevented by decreasing cholesterol levels in blood. Also, when thrombolytic activity, platelet aggregation activity, and excessive blood coagulation tendency are suppressed by inhibiting reactive oxygen species mainly responsible for LDL peroxidation that may cause vascular endothelial cell damage and blood clotting resulting therefrom, it is possible to effectively prevent and improve vascular and circulatory disorders, such as arteriosclerosis, high blood pressure, ischemic heart diseases, and cerebrovascular accident, thereby improving cardiovascular diseases.

The term “metabolic syndrome” refers to a syndrome that involves a collection of various risk factors, such as hyperlipidemia, high blood pressure, impaired glucose metabolism, and obesity. In recent years, this syndrome has been formally termed “metabolic syndrome” or “insulin resistance syndrome” through ATP III (Adult Treatment Program III) established by the WHO (World Health Organization) and the National Heart, Lung, and Blood Institute of the U.S. NIH (National Institutes of Health).

According to the ATP III of the U.S. NCEP (National Cholesterol Education Program), which was published in 2001, metabolic syndrome is diagnosed when one patient has at least three of five risk factors: \textcircled{1} abdominal obesity in which the waist circumference measures more than 40 inches (102 cm) for men and more than 35 inches (88 cm) for women; \textcircled{2} high triglyceride levels of more than 150 mg/dl; \textcircled{3} HDL cholesterol of less than 40 mg/dl for men and less than 50 mg/dl for women; \textcircled{4} high blood pressure of 130/85 mmHg or higher; and \textcircled{5} fasting glucose levels of more than 110 mg/dl. In the case of the Orientals, the abdominal obesity is slightly adjusted to 90 cm for man and 80 cm for woman, and recent research reported that about 25% of Korean population develop symptoms of metabolic syndrome when the above definitions are applied.

Insulin resistance refers to a phenomenon in which although insulin secretion in the body is normal, insulin cannot properly function to supply glucose into cells. Since glucose in blood cannot enter into cells, hyperglycemia emerges, and cells are incapable of their functions due to glucose deficiency, which results in metabolic syndrome. So far, drugs for treating metabolic syndrome have not been developed, and an attempt has been made to treat metabolic syndrome only by using diabetes, hyperlipidemia, and high blood pressure drugs. However, their availability as drugs are limited. Among currently available drugs, metformin, TZD (thiazolidinediones)-based drugs, glucosidase inhibitors, dual PPAR γ/αagonist, and DDP (dipeptidyl peptidase) used for diabetes treatment are in the spotlight as metabolic syndrome drugs. In addition to this, attention is paid to apoA-I isoform and related peptides that are the targets of antihypertensive agents and antihyperlipidemic agents, CETP (cholesterol ester transport protein) inhibitors, and the like.

In the present invention, diabetes, hyperlipidemia, fatty liver, and diseases known to be caused by them, that is, arteriosclerosis, high blood pressure, and cardiovascular diseases, and metabolic syndrome accompanied by simultaneous and multiple occurrence of the above diseases are collectively referred to as “metabolic diseases”.

### DISCLOSURE OF INVENTION

### Technical Problem

Accordingly, the present invention has been made to solve at least the above-mentioned problems occurring in the prior art, and an object of the present invention is to provide raw materials, functional foods, cosmetics, and herb medicines for use as a hepatic protector and a remedy for preventing and treating metabolic diseases, which comprise, as an effective ingredient, a Lysimachiae Foenum-Graeci Herba extract efficacious for reducing glucose levels, triglyceride levels, cholesterol levels, and liver enzyme levels (AST/ALT) in blood, and liver fat.

### Technical Solution

The present invention is characterized by an extract obtained from Lysimachiae Foenum-Graeci Herba efficacious for reducing metabolic disease marker levels (glucose levels, triglyceride levels, cholesterol levels, and liver enzyme levels in blood) and fat in liver tissues.

The present invention provides a method of preparing a Lysimachiae Foenum-Graeci Herba extract efficacious for reducing metabolic disease marker levels (glucose levels, triglyceride levels, cholesterol levels, and liver enzyme levels in blood) and fat in liver tissues by separating the extract from Lysimachiae Foenum-Graeci Herba, the method including the steps of (1) drying and crushing whole plants or leaves of Lysimachiae Foenum-Graeci Herba; (2) subjecting the Lysimachiae Foenum-Graeci Herba obtained in step (1) to solvent extraction by adding an organic solvent thereto in an amount of 5 to 50 times as much as the weight of the Lysimachiae Foenum-Graeci Herba; and (3) filtering an extraction solution of the organic solvent by using a filtration paper, and then subjecting the filtered solution to vacuum concentration at a temperature of 40° C. or less.

The organic solvent includes low alcohol having 1 to 4 carbon atoms.

Since the Lysimachiae Foenum-Graeci Herba extract of the present invention, provided according to the above method, effectively reduces metabolic disease marker levels (glucose levels, triglyceride levels, cholesterol levels, and liver enzyme levels in blood) and fat in liver tissues, it can be used for raw materials, functional foods, and herb medicines for preventing and treating metabolic diseases.

In an embodiment of the present invention, the effect of reducing metabolic disease marker levels (glucose levels, triglyceride levels, cholesterol levels, and liver enzyme levels in blood) and fat in liver tissues was observed by administering the extract of the present invention to patients.

Also, the present invention provides a composition comprising the above extract in an amount of 0.1 to 50% by weight, based on the total weight of the composition. The inventive composition comprising the Lysimachiae Foenum-Graeci Herba extract may further comprise appropriate carriers, excipients, or diluents in a conventional way. Examples of the carriers, excipients, and diluents that may be included in the composition of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinyl pyrrolidone, water, methylhydroxybenzoate, propylhydroxybenzoate, talc, magnesium stearate, and mineral oil. The composition comprising the inventive extract may be used in oral dosage forms, such as powders, granules, tablets, capsules, suspensions, emulsions, syrups, and aerosols, or in the form of external preparations, suppositories, or sterile intravenous infusions in a conventional way. More specially, the composition may be formed into pharmaceutical preparations by using commonly used diluents or excipients, such as fillers, extenders, binders, humectants, disintegrants, and surfactants. Solid preparations for oral administration, including tablets, pellets, powders, granules, capsules, etc., may be prepared by mixing at least one excipient, for example, starch, calcium carbonate, sucrose, lactose, and gelatin, with the inventive extract. Also, lubricants, such as magnesium stearate and talc, may be used in addition to simple excipients. Liquid preparations for oral administration, including suspensions, solutions, emulsions, syrups, etc., may include various excipients, such as humectants, sweeteners, fragrances, and preservatives, in addition to commonly used simple diluents, that is, water and liquid paraffin. Preparations for parenteral administration includes sterile aqueous solutions, non-aqueous solvents, suspensions, emulsions, lyophilized preparations, and suppositories. Propylene glycol, polyethylene glycol, vegetable oil, such as olive oil, injectable esters, such as ethyl oleate, and so forth may be used as non-aqueous solvents and suspensions. Witepsol, macrogol, tween 61, cacao butter, laurin butter, glycerogelatin may be used as bases for suppositories. Although the dosage is different depending on ages, sexes and weights of patients, the extract of the present invention may be generally administrated one to several times per day in an amount of 0.01 to 500 mg/kg and preferably 0.1 to 100 mg/kg. Also, the dosage may be increased or decreased depending on administration routes, seriousness of diseases, sexes, weights, and ages. Accordingly, the scope of the present invention should not be limited to the above dosage in any way.

The composition of the present invention may be administered to mammals including rats, mice, domestic animals, human beings, and the like in various routes. All types of administration routes may be expected, and the composition may be administered with, for example, oral, rectum, or vein, muscle, hypodermic, and intrauterine dura mater or intracerebroventricular injections. Since the Lysimachiae Foenum-Graeci Herba extract of the present invention has almost no toxicity and side effects, it can be safely used even when administered over a long time period for the prevention purpose.

Also, the present invention provides health functional foods for preventing cerebral nervous system-related anxieties, which comprise the Lysimachiae Foenum-Graeci Herba extract and sitologically acceptable food supplement additives. The inventive composition comprising the Lysimachiae Foenum-Graeci Herba extract may be diversely used for drugs, foods, and beverages for preventing cerebral nervous system-related anxieties. Examples of foods to which the extract of the present invention may be added include various beverages, gums, teas, vitamin compounds, health supplement foods, etc., and theses foods may be in the form of pellets, powders, granules, infusions, tablets, or beverages. In general, the extract of the present invention may be added to the health food composition of the present invention in an amount of 0.01 to 15% by weight, based on the total weight of the food composition, and may be added to the health beverage composition of the present invention in an amount of 0.02 to 10 g and preferably 0.3 to 1 g, based on 100 ml of the beverage composition.

Food supplement additives as defined herein include food additive well know in the art, such as sweeteners, flavors, colorants, fillers, and stabilizers. There is no particular limitation on additional ingredients that are added in the health beverage composition of the present invention in addition to a specified amount of the extract as an essential ingredient, and the health beverage composition of the present invention may contain additional ingredients, such as sweeteners or natural carbohydrates, in the same manner as ordinary beverages. Examples of the natural carbohydrates include sugars including monosaccharides (e.g. glucose, fructose, etc.), disaccharides (e.g. maltose, sucrose, etc.), and polysaccharides (e.g. dextrin, cyclodextrin, etc.); and sugar alcohols including xylitol, sorbitol, and erythritol. Natural sweeteners (thaumatin, stevia extracts (e.g. rebaudiocide A, glycyrrhizin, etc.)) and synthetic sweeteners (saccharin, aspartame, etc.) may be advantageously used as sweeteners other than the above ones. The natural carbohydrates may be generally contained in an amount of about 1 to 20 g and preferably about 5 to 12 g, based on 100 ml of the composition of the present invention.

In addition to the above additional ingredients, the composition of the present invention may contain various nutrients, vitamins, minerals (electrolytes), flavors including natural and synthetic flavors, colorants and enhancers (cheeses, chocolates, etc.), pectic acid and salts thereof, alginic acid and salts thereof, organic rid, protective colloid thickening agents, pH modifiers, stabilizers, antiseptics, glycerin, alcohols, carbonating agents used for carbonated beverages, and so forth. In addition, the composition of the present invention may contain fruit flesh for preparing natural fruit juices, fruit juice beverages, and vegetable beverages. Each of these ingredients may be used alone or in combination with other ingredients. The amount of the additives is of little importance, but is generally selected within a range of 0 to 20 parts by weight, based on 100 parts by weight of the composition of the present invention.

### ADVANTAGEOUS EFFECTS

As described in the forgoing, the Lysimachiae Foenum-Graeci Herba extract according to the present invention has a superior effect of reducing metabolic disease marker levels (glucose levels, triglyceride levels, cholesterol levels, and liver enzyme levels in blood) and fat in liver tissues, it can be effectively used for raw materials, functional foods, and herb medicines as a hepatic protector and a remedy for preventing and treating diabetes, hyperlipidemia, fatty liver, and metabolic syndrome accompanied by simultaneous and multiple occurrence of these diseases.

### BRIEF DESCRIPTION OF DRAWINGS

The above and other objects, features and advantages of the present invention will become more apparent from the following detailed description taken in conjunction with the accompanying drawings, in which:

FIG. 1 is a series of pictures illustrating liver tissues extracted from mice of a control group, a case group, and a positive control group, liver tissues stained with hematoxylin-eosin, and fat stained with oil red 0, which is distributed in the liver tissues;

FIG. 2 is graphs illustrating results of measuring the contents of triglycerides and cholesterol in liver tissues by using livers extracted from mice of a control group, a case group, and a positive control group;

FIG. 3 is a graph illustrating results of measuring the amount of glucose uptake by administering the Lysimachiae Foenum-Graeci Herba extract and metformin to mouse myoblast cells (C2C12);

FIG. 4 is a graph illustrating results of measuring the amount of insulin secretion by administering the Lysimachiae Foenum-Graeci Herba extract to hamster pancreatic β-cells (HIT-T15); and

FIG. 5 is a graph illustrating results of measuring the amount of glucose in blood by administering the Lysimachiae Foenum-Graeci Herba extract to diabetic model db/db mice.

### MODE FOR THE INVENTION

Hereinafter, preferred examples and experimental examples of the present invention will be described with reference to the accompanying drawings. However, the following examples are merely illustrative, and the scope of the present invention is not limited thereto.

### EXAMPLES

### Example 1

### Separation of Lysimachiae Foenum-Graeci Herba Extract

Whole plants or leaves of Lysimachiae Foenum-Graeci Herba were dried and crushed, alcohol (methanol or ethanol) was added to the Lysimachiae Foenum-Graeci Herba in an amount of 5 to 50 times as much as the weight of the Lysimachiae Foenum-Graeci Herba, and then active substances were extracted through reflux extraction for 24 hours or more. Subsequently, the alcohol (methanol or ethanol) extraction solution was filtered using a filtration paper, and then was subjected to vacuum concentration at a temperature of 40° C. or less.

### Example 2

### Verification of Effect of Metabolic Disease Prevention and Treatment in Diet-Induced Obesity (DIO) Mice

Seven-week-old C57BL/6 male mice (obtained from Coretec Inc.) were bred in an animal facility where breeding conditions of temperature (20±1° C.), humidity (50±10%), light/dark cycle (12 hours), light intensity (150 to 300 Lux), and ventilation (10 to 20 times/hr) were maintained. Solid feed (obtained from Folas International Inc.) was freely supplied to the mice, and city water was subjected to steam sterilization and then was freely fed to the mice as drinking water. After acclimation for one week, 60% Kcal solid feed (obtained from Research Diet Inc.) was supplied to the mice, and the Lysimachiae Foenum-Graeci Herba extract was orally administered to the mice for 42 days in an amount of 30, 100, and 300 mg/kg/day. Sibutramine was administered to a positive control group in an amount of 10 mg/kg/day, and 0.5% MC (methyl cellulose) was administered to a negative control group in the same amount. The mice to which the substances were administered for six weeks were fasted for one day, and then were killed using CO₂ gas. Blood gathered from the mouse abdominal vena cava was put into EDTA tubes and stored in ice, and then plasma obtained by centrifugation at 3000 rpm for 10 minutes was analyzed using a biochemical auto analyzer (AU400, Olympus, Japan). Also, organs extracted from the mice were immediately frozen by putting them into liquid nitrogen, were stored in a low-temperature refrigerator (−70° C.), and were histologically analyzed.

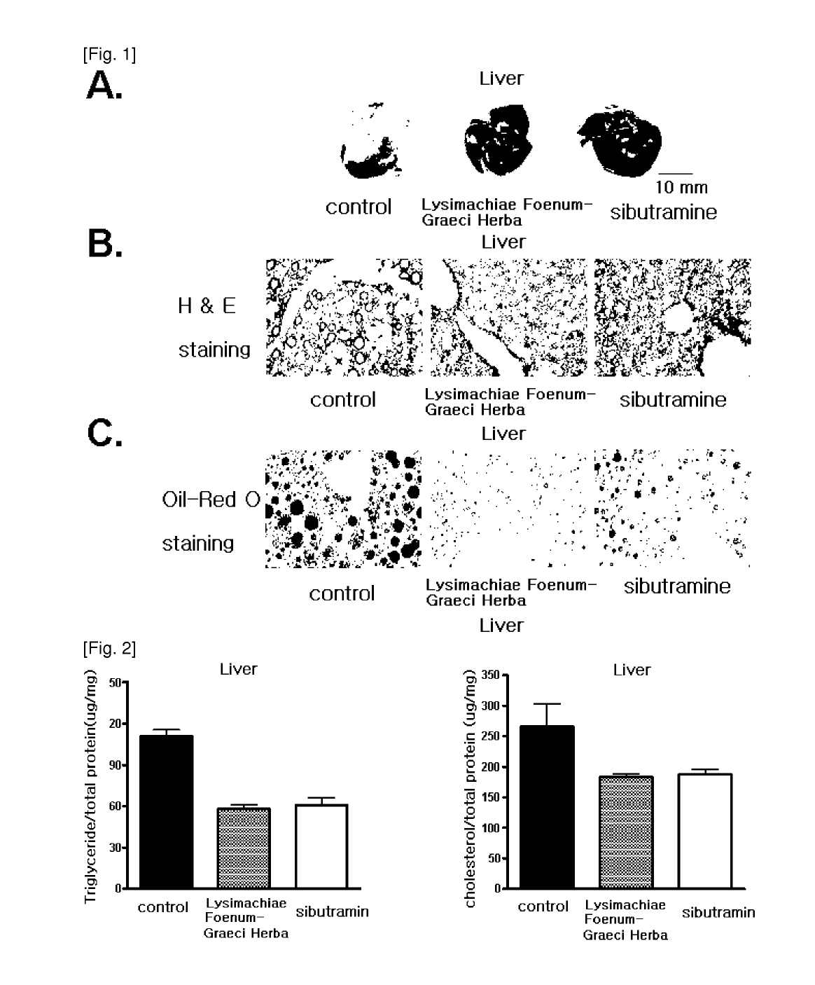

A of FIG. 1 illustrates a picture of livers extracted from the control group, the case group, and the positive control group, and B of FIG. 1 illustrates a picture of histological observations of liver tissues obtained by subjecting the livers extracted from the control, case, and positive control groups to cryosection and then staining the cryosections with hematoxylin-eosin. C of FIG. 1 illustrates a picture of histological observations of fat distributed in the liver tissues, which was obtained by subjecting the livers extracted from the control, case, and positive control groups to cryosection and then staining fat in the cryosections with oil-red O. It could be confirmed from FIG. 1 that the control group exhibited fatty liver because excessive fat accumulation in hepatocytes was caused by high-fat diet, but the case group to which the Lysimachiae Foenum-Graeci Herba extract was administered exhibited a significant improvement in fat accumulation in hepatocytes, as compared to the positive control group.

Table 1 as presented below shows the analysis results of blood gathered for measuring metabolic disease marker levels for DIO (Diet Induced-Obesity) mice. The contents of triglycerides and cholesterol in the extracted liver tissues were also measured, the results of which are given in FIG. 2.

TABLE 1

| | High-fat diet | | | | |
|---|---|---|---|---|---|
| | | | Lysimachiae | Lysimachiae | Lysimachiae |
| | | | Foenum- | Foenum- | Foenum- |
| | | | Graeci | Graeci | Graeci |
| | | Sibutramine | Herba | Herba | Herba |
| | Vehicle | (10 mg/kg) | (30 mg/kg) | (100 mg/kg) | (300 mg/kg) |
| AST | 149.33 ± 29.7 | 172.33 ± 58.4 | 99 ± 13.2 ** | 97.67 ± 17.6 ** | 101.67 ± 11.6 ** |
| (IU/L) | | | | | |
| ALT | 93 ± 22.0 | 64 ± 15.1 * | 54.67 ± 23.8 * | 45.33 ± 18.8 ** | 49.3 ± 10.4 ** |
| (IU/L) | | | | | |
| GLU | 470 ± 90.0 | 182.66 ± 27.3 *** | 213.33 ± 58.5 *** | 268.66 ± 69.9 ** | 193.3 ± 52.6 ** |
| (mg/dl) | | | | | |
| CHO | 172.67 ± 13.6 | 187.33 ± 15 | 154 ± 6.4 * | 151.7 ± 11.7 * | 147 ± 52.3 |
| (mg/dl) | | | | | |
| TG | 72 ± 8.9 | 91.33 ± 17.0 * | 50 ± 10.9 ** | 43.67 ± 10.3 *** | 37.7 ± 12.6 *** |
| (mg/dl) | | | | | |
| ALB | 2.17 ± 0.2 | 2.1 ± 0.1 | 2.2 ± 0.1 | 2.17 ± 0.1 | 2.17 ± 0.2 |
| (g/dl) | | | | | |

Remark: AST (Aspartate aminotransferase), ALT (Alanine aminotransferase), GLU (Glucose), CHO (Cholesterol), TG (Triglyceride), ALB (Albumin)

It could be confirmed from Table 1 that the administration of the Lysimachiae Foenum-Graeci Herba extract statistically meaningfully reduced a rise in liver enzyme AST and ALT, blood glucose, cholesterol, and triglyceride levels, due to high-fat diet. However, in the case of the positive control group to which sibutramine was administered, the ALT and glucose levels were meaningfully reduced, but the AST and cholesterol levels were not changed, and the triglyceride level was rather increased.

Also, it could be confirmed from FIG. 2 that the contents of triglycerides and cholesterol in the liver were meaningfully reduced in the same manner as the positive control group when the Lysimachiae Foenum-Graeci Herba extract was administered.

### Example 3

### Measurement of Antihyperlipidemic Activity of Lysimachiae Foenum-Graeci Herba Extract

Five-week-old ICR male mice (obtained from Central Lab. Animal Inc.) were bred in an animal facility where breeding conditions of temperature (20±1° C.), humidity (50±10%), light/dark cycle (12 hours), light intensity (150 to 300 Lux), and ventilation (10 to 20 times/hr) were maintained. Solid feed for laboratory animals (obtained from Cargill Agri Purina Inc.) was freely supplied to the mice, and city water was subjected to steam sterilization and then was freely fed to the mice as drinking water. After acclimation for one week, the Lysimachiae Foenum-Graeci Herba extract was orally administered to eight mice per group in a concentration of 100 mg/kg, and a saline was orally administered to the control group instead of the extract. After two hours elapsed, 1 g/kg of corn oil was orally administered to the control group, and a saline was orally administered to the normal group instead of corn oil. 2 hours and 30 minutes after the administration of corn oil, blood gathered from the mouse abdominal vena cava was put into EDTA tubes and stored in ice, and then plasma obtained by centrifugation at 3000 rpm for 10 minutes was analyzed using a biochemical auto analyzer (AU400, Olympus, Japan). The analysis results are presented below in Table 2. 10 mg/kg of xenical was used as a control drug.

TABLE 2

| | | | Lysimachiae | |
|---|---|---|---|---|
| | | | Foenum-Graeci | |
| | | | Herba extract | |
| | Normal | Control | (100 mg/kg) | Xenical (10 mg/kg) |
| TG (mg/dl) | 238.80 ± 28.88 | 485.17 ± 55.09* | 300.20 ± 42.70* | 311.17 ± 45.56* |
| CHO (mg/dl) | 147 ± 2.65 | 150.67 ± 2.08 | 145.20 ± 4.49 | 148.83 ± 8.26 |

Remark:
TG (Triglyceride),
CHO (Cholesterol)

As seen from Table 2, when the Lysimachiae Foenum-Graeci Herba extract was administered, the amounts of triglycerides and cholesterol were reduced, as compared when the control drug was used. From this, the antihyperlipidemic activity of the Lysimachiae Foenum-Graeci Herba extract was confirmed.

### Example 4

### Measurement of Antidiabetic Effect of Lysimachiae Foenum-Graeci Herba Extract

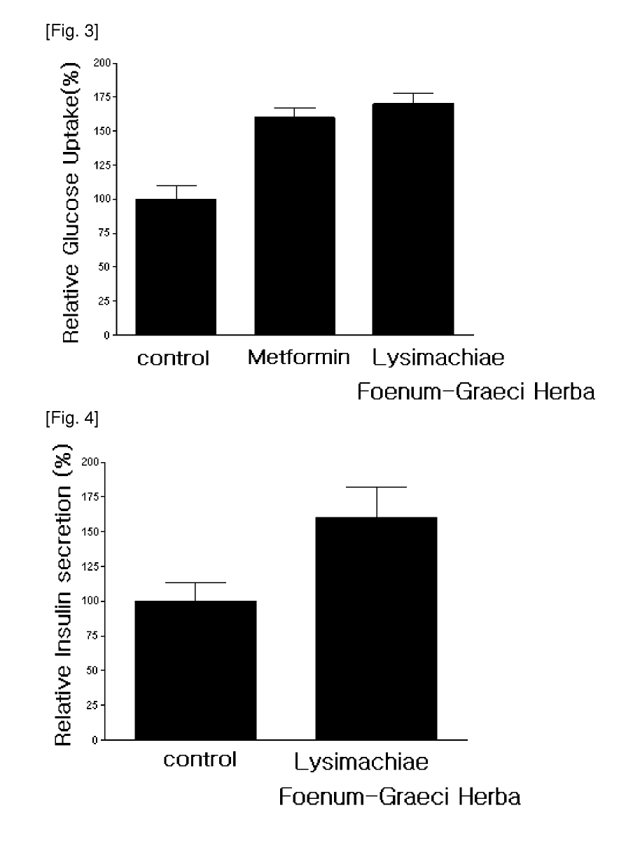

Mouse C2C12 myoblast cells (ATCC, CRL-1772) were cultured in DMEM containing 10% bovine calf serum. When the cell density was about 85 to 90%, cell differentiation was induced by replacing DMEM by 1% bovine calf serum medium. The differentiated C2C12 cells were subjected to starvation by DMEM with low glucose, and then were treated with the Lysimachiae Foenum-Graeci Herba extract in a concentration of 10 ug/ml for 4 hours. Subsequently, the cells were additionally cultured for 20 minutes by exchange with an HEPES buffered saline and treatment with 2-[3H] DG (deoxyglucose). After the HEPES buffered saline was removed from the cells, the cells were washed three times with ice-cold PBS and were dissolved using 0.1N NaOH, and then the level of glucose uptake into the cells was quantitatively analyzed by using a liquid scintillation counter to measure cpm. Metformin (2 mM) was used as a control drug. As a result of the experiment, the amount of glucose uptake was increased by the administration of the Lysimachiae Foenum-Graeci Herba extract, as compared to the control group or the metformin-administered group (FIG. 3).

Also, HIT-T15 pancreatic cells (ATCC, CRL-1777) were treated with the Lysimachiae Foenum-Graeci Herba extract in a concentration of 10 ug/ml for 1 hour, and then the amount of insulin secretion from the cells was determined using an ELISA method, the results of which are illustrated in FIG. 4. As seen from FIG. 4, the amount of insulin secretion was increased by the administration of the Lysimachiae Foenum-Graeci Herba extract, as compared to the control group.

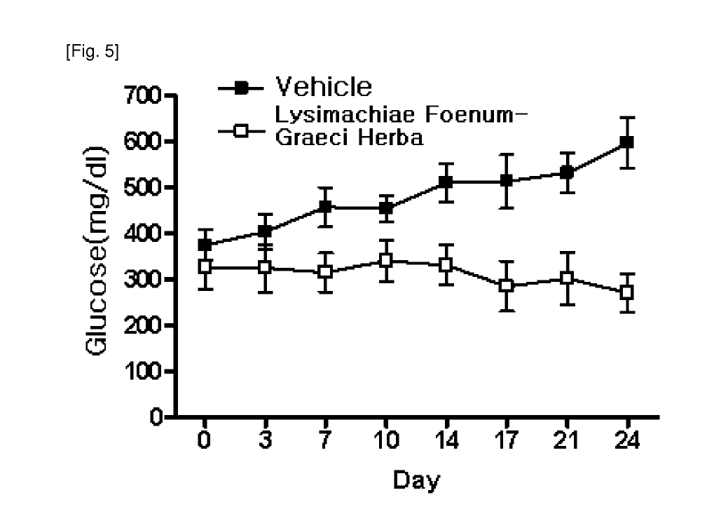

Five-week-old diabetic model db/db male mice (obtained from Central Lab. Animal Inc.) were bred in an animal facility where breeding conditions of temperature (20±1° C.), humidity (50±10%), light/dark cycle (12 hours), light intensity (150 to 300 Lux), and ventilation (10 to 20 times/hr) were maintained. Solid feed for laboratory animals (obtained from Cargill Agri Purina Inc.) was freely supplied to the mice, and city water was subjected to steam sterilization and then was freely fed to the mice as drinking water. After acclimation for one week, the Lysimachiae Foenum-Graeci Herba extract was orally administered to six mice per group in a concentration of 100 mg/kg, and a saline was orally administered to the control group instead of the extract. Blood was gathered from the mouse tail vein twice per week for 24 days, and blood glucose levels in the gathered blood were measured, the results of which are illustrated in FIG. 5. As seen from FIG. 5, the amount of glucose was reduced when the Lysimachiae Foenum-Graeci Herba extract was administered.

From the above experimental results, the antidiabetic effect of the inventive Lysimachiae Foenum-Graeci Herba extract was verified.

### Example 5

### Measurement of Anti-Atherogenic and Anti-Cardiovascular Disease Activities of Lysimachiae Foenum-Graeci Herba Extract

Five-week-old ICR male mice (obtained from Central Lab. Animal Inc.) were bred in an animal facility where breeding conditions of temperature (20±1° C.), humidity (50±10%), light/dark cycle (12 hours), light intensity (150 to 300 Lux), and ventilation (10 to 20 times/hr) were maintained. Solid feed for laboratory animals (obtained from Cargill Agri Purina Inc.) was freely supplied to the mice, and city water was subjected to steam sterilization and then was freely fed to the mice as drinking water. After acclimation for one week, the Lysimachiae Foenum-Graeci Herba extract was orally administered to six mice per group in a concentration of 100 mg/kg for 5 weeks while high-cholesterol diet (Research Diet Inc.) was freely fed to the mice, and a saline was orally administered to the control group instead of the extract. The mice were fasted for 16 hours after the final administration, and then blood gathered from the mouse abdominal vena cava was put into EDTA tubes and stored in ice. Subsequently, plasma obtained by centrifugation at 3000 rpm for 10 minutes was analyzed using a biochemical auto analyzer (AU400, Olympus, Japan), the results of which are presented below in Table 3.

TABLE 3

| | | | Lysimachiae |
|---|---|---|---|
| | | | Foenum-Graeci |
| | | | Herba extract |
| | Normal | Control | (100 mg/kg) |
| TG (mg/dl) | 54.8 ± 4.5 | 100.2 ± 5.3 | 91.4 ± 4.7 |
| CHO (mg/dl) | 83.1 ± 6.2 | 183.7 ± 4.8 | 121.2 ± 3.9 |
| HDL (mg/dl) | 40.8 ± 3.5 | 28.2 ± 1.9 | 33.1 ± 4.8 |
| LDL (mg/dl) | 37.5 ± 2.7 | 80.5 ± 2.0 | 59.6 ± 2.3 |
| Atherogenic index | 1.04 | 5.51 | 2.66 |

Remark:
TG (Triglyceride),
CHO (Cholesterol),
LDL (Low-Density Lipoprotein),
HDL (High-Density Lipoprotein)
Atherogenic index = (total amount of cholesterol amount of HDL cholesterol)/amount of HDL cholesterol

As seen from Table 3, triglyceride levels, the total amount of cholesterol, and LDL levels were reduced, and HDL levels are increased. Accordingly, the atherogenic index was increased.

Reference will now be made to formulation examples for the composition of the present invention.

### Formulation Example 1

### Preparation of Powders

Dry powders of Lysimachiae Foenum-Graeci Herba extract of Example 1: 300 mg

Lactose: 100 mg

Talc: 10 mg

The above ingredients were mixed and filled in an air-tight pack to prepare powders.

### Formulation Example 2

### Preparation of Tablet

Dry powders of Lysimachiae Foenum-Graeci Herba extract of Example 1 50 mg

Corn starch: 100 mg

Lactose: 100 mg

Magnesium stearate: 2 mg

The above ingredients were mixed, and then were subjected to tabletting according to any typical tablet preparation method to prepare a tablet.

### Formulation Example 3

### Preparation of Capsule

Dry powders of Lysimachiae Foenum-Graeci Herba extract of Example 1 50 mg

Corn starch: 100 mg

Lactose: 100 mg

Magnesium stearate: 2 mg

The above ingredients were mixed and filled in a gelatin capsule according to any typical capsule preparation method to prepare a capsule.

### Formulation Example 4

### Preparation of Infusion

Dry powders of Lysimachiae Foenum-Graeci Herba extract of Example 1: 50 mg

Sterile distilled water for injection: Appropriate quantity

pH modifier: Appropriate quantity

An infusion was prepared according to any typical infusion preparation method in the above composition per ampoule (2 ml).

### Formulation Example 5

### Preparation of Liquid Formulation

Dry powders of Lysimachiae Foenum-Graeci Herba extract of Example 1: 100 mg

Glucose isomerase: 10 g

Mannitol: 5 g

Purified water: Appropriate quantity

According to any typical a liquid formulation preparation method, each of the above ingredients was added, dissolved, and mixed in purified water while adding lemon incense in an appropriate amount. Subsequently, purified water was added to the mixture to adjust the total amount to 100 ml, and then the solution was filled and sterilized in a brown bottle to prepare a liquid formulation.

### Formulation Example 6

### Preparation of Health Food

Dry powders of Lysimachiae Foenum-Graeci Herba extract of Example 1: 1000 mg

Vitamin mixture: Appropriate quantity

Vitamin A acetate: 70 μg

Vitamin E: 1.0 mg

Vitamin B1: 0.13 mg

Vitamin B2: 0.15 mg

Vitamin B6: 0.5 mg

Vitamin B12: 0.2 μg

Vitamin C: 10 mg

Biotin: 10 μg

Nicotinic acid amide: 1.7 mg

Folic acid: 50 μg

Calcium pantothenate: 0.5 mg

Inorganic mixture: Appropriate quantity

Iron(I) sulfate: 1.75 mg

Zinc oxide: 0.82 mg

Magnesium carbonate: 25.3 mg

Monobasic potassium phosphate: 15 mg

Dibasic calcium phosphate: 55 mg

Potassium citrate: 90 mg

Calcium carbonate: 100 mg

Magnesium chloride: 24.8 mg

Although the vitamins and the mineral mixture were mixed in a composition ratio comparatively suitable to a health food according to a preferred embodiment, any change may be made to the mixing ratio. The above ingredients were mixed, the granules were prepared according to any typical health food preparation method. The granules prepared in this way may be used for preparing a health food composition according to any typical method.

### Formulation Example 7

### Preparation of Health Beverage

Dry powders of Lysimachiae Foenum-Graeci Herba extract of Example 1: 1000 mg

Citric acid: 1000 mg

Oligosaccharide: 100 g

Concentrated plum juice: 2 g

Taurine: 1 g

Total amount after purified water addition: 900 ml

According to any typical health beverage preparation method, the above ingredients were mixed, and then were stirred and heated at a temperature of 85° C. for one hour to prepare a solution. The prepared solution was filtered and collected in a 2 l sterilized container, and then the solution collected in the container was sealed and sterilized. The obtained solution was stored in a refrigerator, and subsequently was used for preparing a heath beverage composition of the present invention. Although the above ingredients were mixed in a composition ratio comparatively suitable to a fancy beverage according to a preferred embodiment, any change may be made to the mixing ratio depending on regional and racial tastes, such as demanding classes, demanding countries, and uses.

From the above experimental results, the antidiabetic effect of the inventive Lysimachiae Foenum-Graeci Herba extract was verified.

### INDUSTRIAL APPLICABILITY

Since the Lysimachiae Foenum-Graeci Herba extract according to the present invention has a superior effect of reducing metabolic disease marker levels (glucose levels, triglyceride levels, cholesterol levels, and liver enzyme levels in blood) and fat in liver tissues, it can be effectively used for raw materials, functional foods, and herb medicines as a hepatic protector and a remedy for preventing and treating diabetes, hyperlipidemia, fatty liver, and metabolic syndrome accompanied by simultaneous and multiple occurrence of these diseases.

Although preferred embodiments of the present invention have been described in detail for illustrative purposes, those skilled in the art will appreciate that various modifications, additions and substitutions are possible, without departing from the scope and spirit of the invention as disclosed in the accompanying claims.

1. A pharmaceutical composition for preventing and treating at least one metabolic disease selected from the group consisting of hyperlipidemia, fatty liver, diabetes, arteriosclerosis, and cardiovascular diseases, wherein the pharmaceutical composition comprises a Lysimachiae Foenum-Graeci Herba extract as an effective ingredient.

2. The pharmaceutical composition as claimed in claim 1, which is in the form of a capsule, a tablet, a granule, a powder, or a beverage.

3. A health functional food for improving or alleviating symptoms of at least one metabolic disease selected from the group consisting of hyperlipidemia, fatty liver, diabetes, arteriosclerosis, and cardiovascular diseases, wherein the health functional food comprises a Lysimachiae Foenum-Graeci Herba extract as an effective ingredient.

4. A health functional food for improving liver functions, wherein the health functional food comprises a Lysimachiae Foenum-Graeci Herba extract as an effective ingredient.

5. The health functional food as claimed in claim 4, which is in the form of a capsule, a tablet, a granule, a powder, or a beverage.
